(19) United States
(12) Patent Application Publication
(10) Pub. No.: US 2023/0120577 A1
(43) Pub. Date: Apr. 20, 2023

(54) PATCH OF FORMULATION OF RAW CANNABINOIDS AND METHODS

(71) Applicant: Lynne Deupree Yancey, Atlanta, GA (US)

(72) Inventor: Lynne Deupree Yancey, Atlanta, GA (US)

(21) Appl. No.: 17/957,607

(22) Filed: Sep. 30, 2022

Related U.S. Application Data
(60) Provisional application No. 63/251,431, filed on Oct. 1, 2021.

(51) Int. Cl. A61K 36/185; A61K 31/352; A61K 31/05; A61K 31/60; A61K 9/00

(52) U.S. Cl. CPC A61K 36/185; A61K 31/352; A61K 31/05; A61K 31/60; A61K 9/0014; A61K 2236/15

(57) ABSTRACT

The present disclosure provides a raw cannabis composition, the raw cannabis composition comprises a cannabis biomass dispersed in a transdermal carrier, and the cannabis biomass composition comprises an anesthetically effective amount of a cannabinoid acid compound and is substantially free from the cannabinoid acid compound in its decarboxylated form. The cannabinoid acid compound comprises a major cannabinoid acid compound that is tetrahydrocannabinolic acid (“THCA”) and a minor cannabinoid acid compound other than THCA. Methods of making and using the raw cannabis composition are also disclosed.

### CROSS-REFERENCE TO RELATED APPLICATION

This application claims the benefit of U.S. provisional application No. 63/251,431, filed Oct. 1, 2021, the teaching of which is incorporated herein in its entirety by reference.

### TECHNICAL FIELD OF THE DISCLOSURE

This disclosure generally relates to transdermal delivery via a patch of a formulation of raw cannabinoids.

### BACKGROUND OF THE DISCLOSURE

Background information related to the present disclosure as described herein may not constitute prior art.

Methods and products for transdermally administering particular chemicals are known in the art. In the fields of cannabinoids delivery, transdermal delivery has been focused on delivery of ingredients tetrahydrocannabinol (THC), cannabinol (CBN), cannabidiol (CBD) and cannabichromene (CBC). Several medicinal uses have been found for the active ingredients of cannabis, including the ingredients tetrahydrocannabinol (THC), cannabinol (CBN), cannabidiol (CBD) and cannabichromene (CBC). Such medicinal uses of cannabis include (1) treatment of nausea and pain associated with cancer and chemotherapy; (2) nausea, pain and wasting associated with AIDS; (3) arthritis and rheumatism; (4) glaucoma; (5) migraines; (6) muscle spasticity associated with multiple sclerosis and paralysis; (7) alcohol and narcotics withdrawal; (8) stress and depression; (9) asthma; and (10) epileptic seizures. However, transdermal delivery of THC does not include transdermal delivery of acidic form of cannabinoids, such as THCA (tetrahydrocannabinolic acid), a precursor of THC; CBDA (cannabidiolic acid) or CBCA (cannabichromene acid).

Acidic form of cannabinoids has medicinal properties that can be different from those of its decarboxylated counterpart. Therefore, transdermal delivery of an acidic form of cannabinoid is desirable.

The embodiments described below address such issues or problems.

### SUMMARY OF THE DISCLOSURE

In one aspect of the present disclosure, it is provided a raw cannabis composition, comprising a cannabis biomass dispersed in a transdermal carrier, the cannabis biomass composition comprises an anesthetically effective amount of a cannabinoid acid compound and is substantially free from the cannabinoid acid compound in its decarboxylated form, and the cannabinoid acid compound comprises a major cannabinoid acid compound that is tetrahydrocannabinolic acid (“THCA”) and a minor cannabinoid acid compound other than THCA.

In some embodiments of the disclosed composition, optionally in combination with any or all of the various embodiments disclosed herein, the raw cannabis composition further comprises one selected from the group consisting of terpenes, flavonoids and combinations thereof.

In some embodiments of the disclosed composition, optionally in combination with any or all of the various embodiments disclosed herein, the minor cannabinoid compound is one of cannabidioloic acid (“CBDA”), and cannabichromenic acid (“CBCA”), and the decarboxylated form is one of tetrahydrocannabinol (“THC”); cannabidiol, and cannabichromene.

In some embodiments of the disclosed composition, optionally in combination with any or all of the various embodiments disclosed herein, the transdermal carrier comprises an oil or an amount of water.

In some embodiments of the disclosed composition, optionally in combination with any or all of the various embodiments disclosed herein, the transdermal carrier further comprises an enhancer.

In some embodiments of the disclosed composition, optionally in combination with any or all of the various embodiments disclosed herein, the raw cannabis composition is in a formulation of skin patch.

In some embodiments of the disclosed composition, optionally in combination with any or all of the various embodiments disclosed herein, the biomass is in a particulate form, a pressed form, or a grinded form.

In another aspect of the present disclosure, it is provided a method of forming a raw cannabis composition, comprising:

providing a cannabis biomass,
mixing a transdermal carrier with the cannabis biomass, and
forming a transdermal formulation of the raw cannabis composition,

wherein the cannabis biomass composition comprises an anesthetically effective amount of a cannabinoid acid compound and is substantially free from the cannabinoid acid compound in its decarboxylated form, and

wherein the cannabinoid acid compound comprises a major cannabinoid acid compound that is tetrahydrocannabinolic acid (“THCA”) and a minor cannabinoid acid compound other than THCA.

In some embodiments of the disclosed method, optionally in combination with any or all of the various embodiments disclosed herein, the cannabis biomass further comprises one selected from the group consisting of terpenes, flavonoids and combinations thereof.

In some embodiments of the disclosed method, optionally in combination with any or all of the various embodiments disclosed herein, the minor cannabinoid compound is one of cannabidioloic acid (“CBDA”), and cannabichromenic acid (“CBCA”), and the decarboxylated form is one of tetrahydrocannabinol (“THC”); cannabidiol, and cannabichromene.

In some embodiments of the disclosed method, optionally in combination with any or all of the various embodiments disclosed herein, the transdermal carrier comprises an oil or an amount of water.

In some embodiments of the disclosed method, optionally in combination with any or all of the various embodiments disclosed herein, the transdermal carrier further comprises an enhancer.

In some embodiments of the disclosed method, optionally in combination with any or all of the various embodiments disclosed herein, the raw cannabis composition is in a formulation of skin patch.

In some embodiments of the disclosed method, optionally in combination with any or all of the various embodiments disclosed herein, the biomass is in a particulate form, a pressed form, or a grinded form.

In a further aspect of the present disclosure, it is provided a method of use, the method comprises applying to a patient in need thereof a raw cannabis composition that comprises a cannabis biomass dispersed in a transdermal carrier, the cannabis biomass composition comprises an anesthetically effective amount of a cannabinoid acid compound and is substantially free from the cannabinoid acid compound in its decarboxylated form, and the cannabinoid acid compound comprises a major cannabinoid acid compound that is tetrahydrocannabinolic acid (“THCA”) and a minor cannabinoid acid compound other than THCA.

In some embodiments of the disclosed method, optionally in combination with any or all of the various embodiments disclosed herein, the cannabis biomass further comprises one selected from the group consisting of terpenes, flavonoids and combinations thereof.

In some embodiments of the disclosed method, optionally in combination with any or all of the various embodiments disclosed herein, the minor cannabinoid compound is one of cannabidioloic acid (“CBDA”), and cannabichromenic acid (“CBCA”), and the decarboxylated form is one of tetrahydrocannabinol (“THC”); cannabidiol, and cannabichromene.

In some embodiments of the disclosed method, optionally in combination with any or all of the various embodiments disclosed herein, the transdermal carrier comprises an oil or an amount of water

In some embodiments of the disclosed method, optionally in combination with any or all of the various embodiments disclosed herein, the transdermal carrier further comprises an enhancer.

In some embodiments of the disclosed method, optionally in combination with any or all of the various embodiments disclosed herein, the raw cannabis composition is in a formulation of skin patch.

In some embodiments of the disclosed method, optionally in combination with any or all of the various embodiments disclosed herein, the biomass is in a particulate form, a pressed form, or a grinded form.

In some embodiments of the disclosed method, optionally in combination with any or all of the various embodiments disclosed herein, the patient suffers from medical condition associated with acute or chronic pain.

### BRIEF DESCRIPTION OF THE DRAWINGS

FIGS. 1 and 2 show a cannabis plant.

FIG. 3 shows an example of separation of cannabis plant into various components, A, B, and C.

FIG. 4A shows the captured cannabis matter filtered through an herb grinder and separated into the smallest particles, known as kief. FIG. 4B shows the combining of small cannabis particles (kief) with generic petroleum jelly or oil to formulate a cream. The mixture, seen in FIG. 5, can then be applied to a wound as demonstrated in FIG. 6, below.

FIG. 5 shows an example of forming a raw cannabis composition of the present disclosure (TheraTri).

FIG. 6 shows an example of using a raw cannabis composition of the present disclosure.

FIG. 7 illustrates an overall process of making and using a raw cannabis composition as shown in FIGS. 1-6.

### DETAILED DESCRIPTION OF THE DISCLOSURE

### Definitions

“Cannabis”, as used herein, means a cannabis solution which has been preferably extracted from its natural source such as marijuana and hashish, or any one or more compound or chemical component thereof, including tetranydrocannabinol (THC), cannabinol (CB), cannabidiol (CBD) and cannabichromene (CBC).

Raw Cannabinoid Acid Composition

In one aspect of the present disclosure, it is provided a raw cannabis composition, comprising a cannabis biomass dispersed in a transdermal carrier, wherein the cannabis biomass composition comprises an anesthetically effective amount of a cannabinoid acid compound and is substantially free from the cannabinoid acid compound in its decarboxylated form.

In some embodiments of the disclosed composition, optionally in combination with any or all of the various embodiments disclosed herein, the raw cannabis composition further comprises one selected from the group consisting of terpenes, flavonoids and combinations thereof.

In some embodiments of the disclosed composition, optionally in combination with any or all of the various embodiments disclosed herein, the cannabinoid acid compound is tetrahydrocannabinolic acid (“THCA”) and the decarboxylated form is tetrahydrocannabinol (“THC”).

In some embodiments of the disclosed composition, optionally in combination with any or all of the various embodiments disclosed herein, the transdermal carrier comprises an oil or an amount of water.

In some embodiments of the disclosed composition, optionally in combination with any or all of the various embodiments disclosed herein, the transdermal carrier further comprises an enhancer.

In some embodiments of the disclosed composition, optionally in combination with any or all of the various embodiments disclosed herein, the raw cannabis composition is in a formulation of skin patch.

In some embodiments of the disclosed composition, optionally in combination with any or all of the various embodiments disclosed herein, the biomass is in a particulate form, a pressed form, or a grinded form.

As used herein, the term “oil” comprises any one or mixture of pharmaceutical grade light mineral oils, vegetable oils, fish and animal oils. Examples of vegetable oils are sesame, corn cottonseed, almond, orange, lemon, eucalyptus, olive, peanut, safflower, cinnamon, clove, coconut and soybean oils. Other usable oils are cod liver and castor oils. The word “structure” means one or more layers of material suitable for attachment to one's skin, including strips or patches of fabric, plastic, metal foil, rubber, resin film, natural membranes and laminates of any one or combination of the above.

Cannabis and Harvest Thereof

Characteristics of a typical cannabis material useful with the disclosure are as described below.

As used herein, the term “TheraTri” refers to and is categorized as a non-synthetic, local anesthetic that does not cause the user or to lose consciousness. TheraTri also in one embodiment has a short-term effectiveness, usually lasting around 30 minutes. In other embodiments, TheraTri can last longer, from more than 30 minutes to about 2 hrs. The TheraTri formula has analgesic properties and uses a combination of naturally occurring cannabinoids, terpenes, flavonoids which are collected in their purest form in the plants' kief. While the type of strain appears not determinative of this phenomenon, but rather neuronal targeting, GABA A receptor interaction, sodium ion channel prohibition; and similar to anesthesia, site alteration integration with the central nervous system and the endocannabinoid system.

At the cellular level, anesthetics alter the behavior of neurons, by interacting directly with a small number of ion channels. Local anesthetics interrupt neural conduction by inhibiting the influx of sodium ions through channels or ionophores within neuronal membranes.

Gamma-Aminobutyric Acid (GABA) is the main ionotropic inhibitory neurotransmitter system (messenger) in the central nervous system (CNS) of vertebrates. GABA A receptors are generally found at inhibitory synapses in the hippocampus and are involved in cellular communication level. The recently discovered endocannabinoid system (ECS) is a lipid signaling network and shows to be abundantly present in GABA-rich areas of the brain. The ECS and GABA A receptors are connected through neuronal chemical messaging in the CNS and communicate through a system of activities at localized synaptic levels.

Currently the ECS has two endocannabinoids that function between neurons as chemical messengers: Anadamide (AEA) and 2-Arachidonyl Glycerol (2-AG). (2-AG) is a signaling lipid in the central nervous system that is a key regulator of neurotransmitter release and an important endocannabinoid that activates the cannabinoid CB1 receptor. TheraTri, a phytocannabinoid-based medicine enters the body at the point of absorption. Phytocannabinoids from TheraTri's formula bind to endogenous CB1 or CB2 cannabinoid receptors, and affect voltage- and ligand-gated ion channels.

CB1 and CB2 receptors are critical to the analgesic reaction that is stimulated when the TheriTri formula is absorbed trandermally into the body because of associated roles with the ECS as G-protein coupled receptors (GPCRs). The pharmaceutical industry target GPCRs because they act as gatekeepers of molecular signals including pain inhibition, inflammation, and immune cell migration.

According to the National Library of Medicine, every anesthesia, at clinical concentrations, modulates the function of two or more types of channels in the Central Nervous System. In conclusion, phytocannabinoids tetrahydrocannabinolic acid (THCA), cannabidioloic acid (CBDA) and cannabichromenic acid (CBCA) used in TheraTri, combined with endocannabinoids (2-AG); modulate the two G Protein-coupled receptors (CB1) and (CB2), which react by stimulating GABA A receptors. This direct molecular interaction between the endocannabinoid (2-AG) increases the power of GABA A receptors, prohibits sodium ion channels which in turn, alters the (CNS) acting as a nerve block at the site of the wound.

Kief and raw plant material taken from sativa dominant strains are just as adaptable as indica dominant strains.

It is important to note TheraTri is comprised of pure and natural components, realizing it does not contain any synthetic cannabinoids. Synthetically made cannabinoids have been documented to be addictive and are notorious for wreaking havoc on the human psyche and body.

Kief

Kief is a term for the crystal-like formations comprised of resinous glands which look like tiny ‘hairs’ called trichomes that grow on the flower of cannabis plants. Trichomes are configured similar to mushrooms with a cylindrical body attached to a spherical head. Trichomes are the plants' resin glands that contain compounds of the cannabis plant including high amounts of terpenes, flavonoids and cannabinoids.

These glandular trichomes are where the highest concentration of phytocannabinoids, like tetrahydrocannabinolic acid (THCA), cannabidioloic acid (CBDA) and cannabichromenic acid (CBCA) exist. When using various extraction methods, the spherical head of the trichrome falls off while the shaft remains attached to the bud. Under close inspection, spherical trichomes look like a powdery substance and can be separated and collected from the plant using an herb grinder or dry sifter screen. Presently, kief is commonly used for inhalable smoking, vaping, cooking or pressed into hash.

### Harvest Time, Trichomes and Kief

Along with the pistils and calyxes, trichomes are an imperative part of the cannabis plant. Trichomes contain the flavor, smell and characteristics of the cannabis plant, i.e. cannabinoids, terpenes and flavonoids. Determining when to harvest the plant depends on the trichomes.

Generally, cannabis seeds come with instructions on estimated flower time (8-9 weeks) but the answer can also be achieved by the color of trichomes which portrays ripeness, or freshness of the plant.:

Underripe: Clear trichomes

Ripe: Milky/cloudy trichomes

Overripe: Amber trichomes

Milky colored ripe trichomes are tricky to catch and require constant supervision and tedious observation down to the hour. Milky color depicts the trichomes are at the height of potency and is important to the TheraTri formula for its need for material with high therapeutic properties. Potency can be determined using a microscope or hand-held loupe to examine trichomes growing on cannabis plants. This technology is common with growers and used to determine when to harvest their cannabis plants.

### Stages of Activation

Stages of activation in the cannabis plant are important to understand TheraTri and how it differentiates from other extractions.

1^{st} stage: Raw, freshly harvested marijuana has nutrients and varieties of cannabinoids in their acidic form (i.e. poplar examples THCA tetrahydrocannabinolic acid and CBDA cannabidiolic acid). The pistils and the calyx are most cannabinoid-dense parts of the plant. Resin is highly concentrated in the calyx and pistil, so this area of the plant that is harvested and sold as cannabis bud (flower).

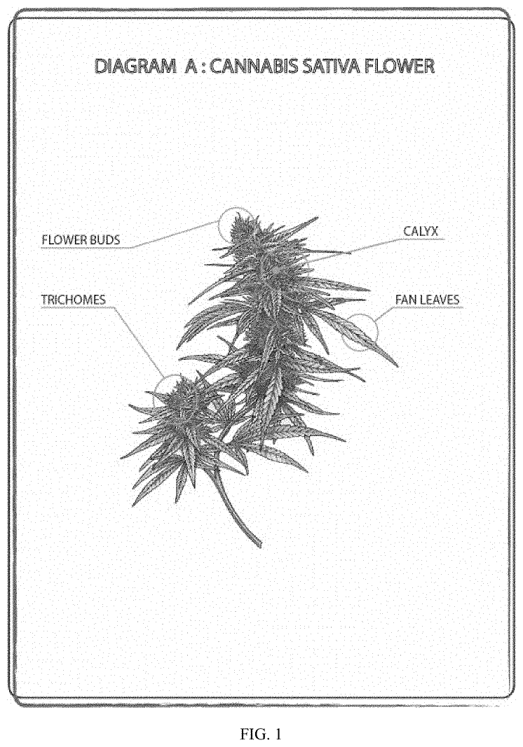

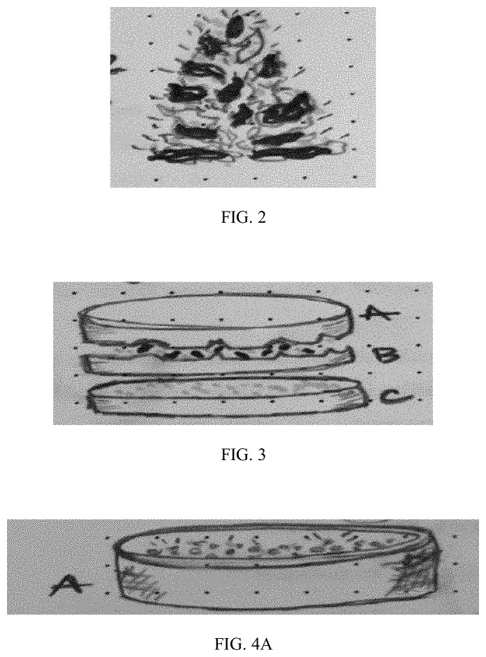

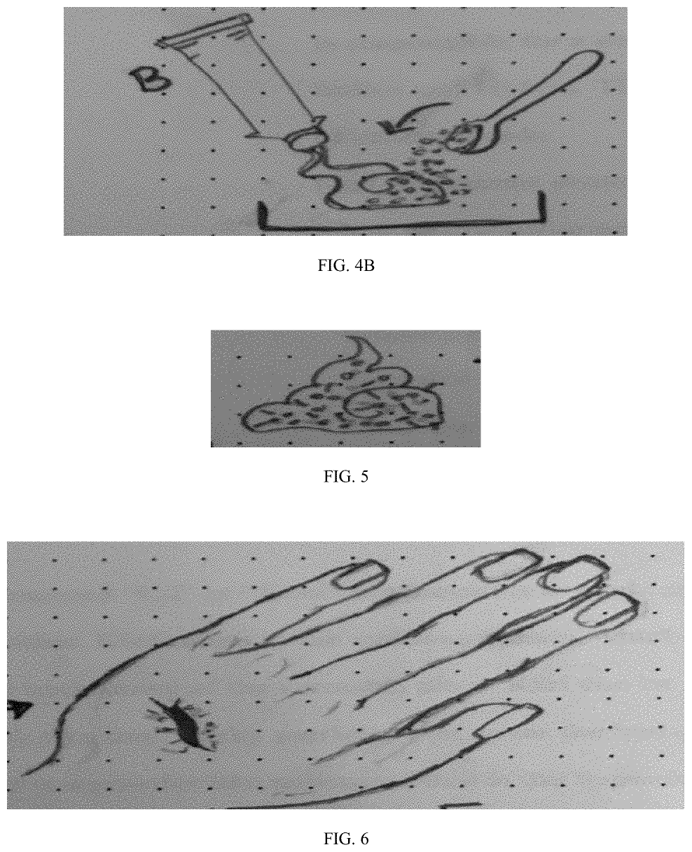

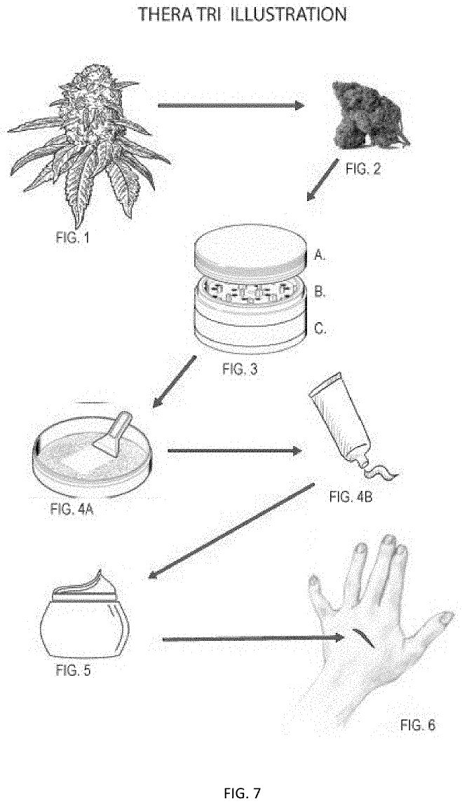

Raw cannabis plant matter (buds and foliage) must be dried and then put though a heating process called decarboxylation in order to activate and convert (i.e. THCA→THC). See FIGS. 1 and 2. 2^{nd} stage: Drying freshly picked marijuana is the original natural process helps prepare the plant for consumption. See FIG. 3. Referring to FIG. 3, a volume of dried cannabis plant (AKA marijuana) is separated using an herb grinder (See A, B, and C components on drawing). An efficient way to separate trichome-potent material from the rest of the plant is to process it through a three-chamber herb grinder. Bud from the cannabis flower is placed between Chambers A and B and ground into small pieces, which fall into the collection chamber below B. The smallest particles containing rich trichomes, the kief, tend to range in size between 75-125 microns and fall into Chamber C, where they can be collected and harvested for use in TheraTri. This kief will be white to yellow to greenish in color and is ideal as it contains plant matter.

In FIGS. 4A, 4B and 5, an amount of each of components A, B, and C is combined with a carrier D to form an anesthetic composition. The TheraTri art has shown that kief will act as an anesthesia but has a higher absorption rate when added to a carrier oil. See FIG. 4 A: Once the resinous kief is separated from cannabis flower buds it can be combined with a carrier oil to help hold it in place. FIG. 4B: Cannabinoid-dense kief mixed with carrier oil can then be placed on a small cut or open wound and is advanced through lipophilic absorption through the skin into the bloodstream; reacting as an anesthesia and effectively numbing the site of the transdermal wound. FIG. 6 shows that a composition (TheraTri) formed by a process shown in FIGS. 3, 4A, 4B, and 5 is applied to a site on a hand of a patient. FIG. 7 illustrates an overall process of making and using a raw cannabis composition as shown in FIGS. 1-3, 4A, 4B, 5 and 6.

Raw cannabis cannot incur a ‘high’ when ingested and to do so the plant must go through several stages of heating to chemically transform and activate its compounds.

Heat from the sun, helps encourage some of the acidic cannabinoids to chemically transform into their activated forms. At this stage there may be slight psychoactive effects and medical benefits. However, current cannabis markets use cannabinoids post-decarboxylation process to produce potent cannabinoids and release full psychoactive effects.

3^{rd} stage: Decarboxylation is achieved through the application of heat. Using THCA as an example, the decarboxylation process implies a chemical removal of the acid radical group CO₂ with high temperatures. Post-decarboxylation, when the acid molecule is removed and THC has been fully activated, ready for consumption via inhalation or digestion.

### Scheme 1

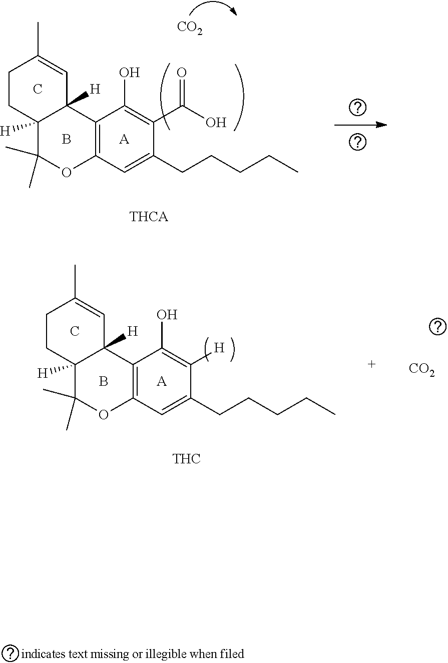

Source: https://honestmarijuana.com/decarboxylation/

We found that raw cannabinoids (particularly stage 2) act with powerful herbal and medical properties. In one embodiment, the raw cannabinoid ingredients include partially raw plant matter from Stage 2, including trichomes (naturally potent with cannabinoids) occurring in kief or resinous part of the plant, affirming they are partly activated and partly raw but have not yet gone through the decarboxylation process. The tip of the trichome is where the highest concentration of the phytocannabinoid, tetrahydrocannabinolic acid (THCA) is stored.

To date, the only cannabis—derived medicine, synthetic or non-synthetic—that we are aware of uses oils which have been decarboxylated. TheraTri does not go through the 3^{rd} activation stage and therefore is unique.

### Chemistry of Cannabis

The process of extraction is commonly employed to obtain target bioactive compounds from complex plant matter.

Commercial cannabis concentrates & extracts are primarily made from extracts containing only trichome dense, activated cannabinoids not any other part of the plant. The preservation of acidic cannabinoids requires the extraction to be made at room temperature (Citti et aL. 2016)

Cannabis extraction can be used to concentrate target components for product development. Important parameters effect the yield and potency of the extract: extraction time, temperature, size and rate of agitation. Solventless and solvent-based are explored below. Two prime categories of trichrome extraction: Mechanical Separation (physical separation) and Chemical extraction (solvent-based extraction).

Mechanical separation can be done a few different ways: most common is shaking the trichomes free or using heat and pressure to dissolve trichomes for separation. For example ice water hash, (bubble hash) which freezes then collects trichomes using a mesh filter. Rosin uses heat and pressure resulting in sticky, caramel-like goo that contains up to 80% THC. However, high pressure at low temperatures is not achievable, therefore retention of terpenes is limited (analytical cannabis.com).

Chemical extraction is quick and uses a solvent to strip plant of its trichomes. Solvent-based extractions have a higher success rate of capturing full spectrum cannabinoids. While most commercial entities prefer solvent extraction, high temps tend to destroy the plant matter therefore chemical extractions usually have less potent cannabinoids if any at all.

Again, TheraTri differs from all the above by using the marijuana plants' partially raw dried material. Together, this combination become the ingredients for TheraTri which originates from non-decarboxylated plant matter, (kief) and its natural compounds including cannabinoids, flavonoids, terpenes and their trichomes. See FIGS. 3-6.

### Sativa Strain Profile Vs Indica Strain Profile

Another important aspect that determines how and why TheraTri is successful as a formula is its use of strains with pain alleviating properties. At present, there are thousands of unique strains with different genetic profiles. To make it easier, I've narrowed down my claim using one sativa and one indica-dominant strains both displaying pain alleviating properties:

Sativa dominant strains with potent cannabinoids with pain alleviating effects similar to Golden Goat are desirable: Full chemical profile

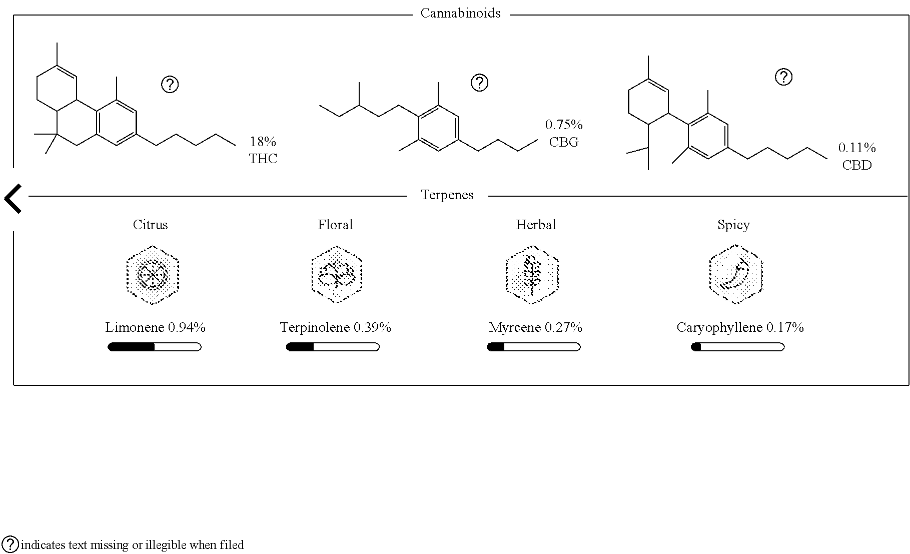

Compared to Skywalker OG an indica-dominant strain with potent cannabinoids and pain alleviating tendencies:

### Full Chemical Profile

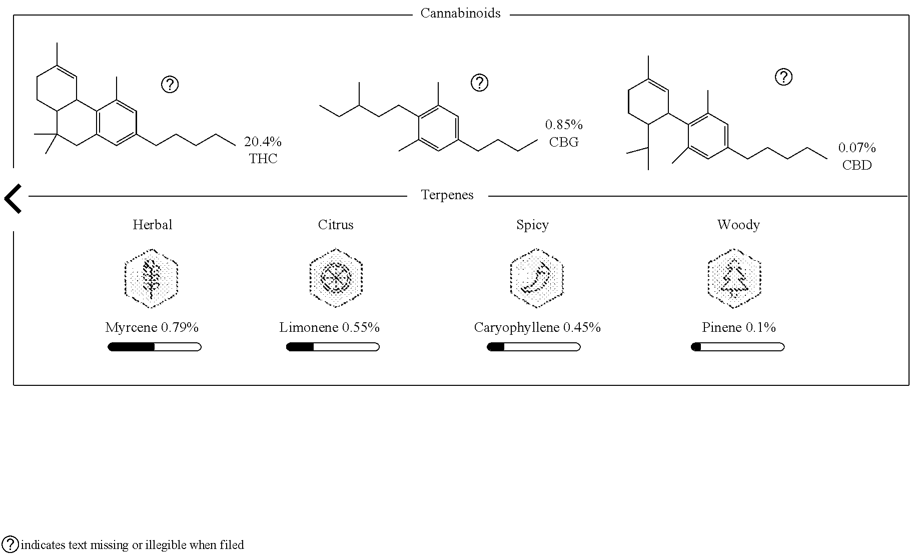

Source: https://cannigma.com/

### Method of Fabrication

In another aspect of the present disclosure, it is provided a method of forming a raw cannabis composition, comprising:

providing a cannabis biomass,
mixing a transdermal carrier with the cannabis biomass, and
forming a transdermal formulation of the raw cannabis composition,

wherein the cannabis biomass composition comprises an anesthetically effective amount of a cannabinoid acid compound and is substantially free from the cannabinoid acid compound in its decarboxylated form.

In some embodiments of the disclosed method, optionally in combination with any or all of the various embodiments disclosed herein, the cannabis biomass further comprises one selected from the group consisting of terpenes, flavonoids and combinations thereof.

In some embodiments of the disclosed method, optionally in combination with any or all of the various embodiments disclosed herein, the cannabinoid acid compound is tetrahydrocannabinolic acid (“THCA”) and the decarboxylated form is tetrahydrocannabinol (“THC”).

In some embodiments of the disclosed method, optionally in combination with any or all of the various embodiments disclosed herein, the transdermal carrier comprises an oil or an amount of water.

In some embodiments of the disclosed method, optionally in combination with any or all of the various embodiments disclosed herein, the transdermal carrier further comprises an enhancer.

In some embodiments of the disclosed method, optionally in combination with any or all of the various embodiments disclosed herein, the raw cannabis composition is in a formulation of skin patch.

In some embodiments of the disclosed method, optionally in combination with any or all of the various embodiments disclosed herein, the biomass is in a particulate form, a pressed form, or a grinded form.

As used herein, the term “skin patch” refers to a topical delivery device for transdermal delivery of a anesthesia agent or a medical agent (e.g., an agent for pain). Various skin patch devices are commercially available. Some examples of such skin patch devices are described in U.S. Pat. No. 6,328,992B1, the teaching of which is incorporated herein by reference in its entirety.

In some embodiments, the raw cannabis composition comprises a liquid or gel carrier combined with the aforementioned oil and the cannabis component(s). The amount of carrier can range from about 10-90 weight percent of the overall preparation and the oil may be present in a range of 1-90 weight percent of the preparation.

Other suitable carriers are natural rubber blends, viscoelastic semi-solids such as pressure sensitive adhesive materials, hydrogels, soft thermoplastic polymers such as ethylene vinyl acetate with high VA contents, elastomers such as polyisoprene elastomers and thermoplastic elastomers such as styrene-butadiene block copolymers.

Other effective gel or liquid carriers may include carbon tetrachloride, ethanolic solutions of resin and pyrahexyl mixed with an amount of the raw cannabis components described above, Tween 80 or petrol ether. In all cases, the carrier material should be inert to the cannabis chemicals and permit easy migration of the preparation to the patient's skin.

The rate controlling means is located directly adjacent to the patient's skin. Its function is to control the flux of cannabis from the reservoir to the skin. A preferred rate controlling means may comprise a polymer membrane having a predetermined permeability and thickness for allowing the release of effective amounts of cannabis continuously from several hours to several days.

Once an appropriate polymer is chosen, the membrane may be formed by preparing a homogenous solution containing the polymer and an organic solvent. The solution is cast upon a glass plate or equivalent, where the solvent is evaporated from the solution. The evaporation of the solvent results in a film which comprises the membrane, and the thickness of the membrane can be varied as required by the desired cannabis flux.

Alternatively, the rate controlling membrane may be in film form.

Some factors to consider in determining an appropriate polymer membrane include the polymer's resistance to deterioration from cannabis, and the polymer's permeability towards cannabis. Previous transdermal applications have used dense nonporous materials such as commercial polyethylene.

Nonporous polymer materials offer the advantage of administering the drug over the greatest period of time. However, nonporous polymer materials are not necessarily optimally suited for a transdermal cannabinoid structure, since cannabis components have relatively large molecular sizes and exhibit unique chemical interactions such as binding with some materials.

Rate controlling may also be achieved using porous materials which are fastened to a backing layer with adhesives, sonic welding or heat-sealing techniques. The cavity is then suspended between the backing layer and the porous material. Prior experimentation has shown that cannabis ingredients diffuse rapidly through certain porous materials such as rubber and plastic.

Furthermore, cannabinolic acid can be insoluble with many solutions, including aqueous and ionically charged solutions. An application of an ionic aqueous solution to a porous material will hinder the diffusion rate of THCA, for example, through the material and decrease flux of cannabis. Therefore, an appropriate combination of porous THCA, for example, absorbing material, combined with a solution that is insoluble with THCA, can form a suitable rate control means. An example of such a rate controlling membrane includes mixing salt water with a porous rubber membrane that covers the cavity. The thickness of the porous rubber membrane, the concentration of the salt water, and the amount of available cannabis in the cavity are optimized experimentally to create a desired flux of THCA, for example, to the patient's skin. Evaporation of necessary fluids may be prevented by a protective backing layer.

Permeation enhancer preparations can be present in an amount of about one to fifty weight percent of the overall cannabis composition. The most effective enhancers are nonionic surfactants or solvents having an HLB value from about 6 to 30. They are selected from chemical groups of glycerol esters, polyglycerol esters, alkyl fatty acid esters, ethoxylated sorbitan esters, alcohol ethoxylates, lanolin ethoxylates, ethoxylated fatty methyl esters and alkanolamides. As used herein, the term “HLB” is a numeric expression of the ability to emulsify non-soluble ingredients in oil and water. It represents the “HydrophileLipophile Balance of an emulsifier.

For a more effective or predictable method of transdermal delivery, the cannabinoid structure may be used in conjunction with an auxiliary means for facilitating a transdermal application. An example of an auxiliary means is the application of a patch containing a low dosage on a portion of the patient's skin containing artificially induced pores, such as those created by pin pricks.

Alternatively, enhanced permeability can be achieved using a chemical carrier that increases the permeability of the user's skin with respect to cannabis. The chemical carrier may be incorporated into the cannabis flux or be administered to the patient's skin as a precedent step to the cannabis application. Examples of suitable carriers include ionically charged materials, such as urea, which polarize the skin's molecules and increase the skin's permeability through ionic force. Another example is a solution of DMSO (dimethyl sulfoxide). This material may be incorporated into the cannabis preparation in volumetric concentrations of up to about ninety percent.

An example of forming the cannabis preparation involves drying and grinding to a fine powder a cannabis plant material. This powder is then refluxed with alcohol or petroleum for three to four hours to separate the cannabis oils from the plant cellulose mass. The resultant extract is further purified and concentrated by removing tars and waxes with an alcohol petroleum ether and water wash. The remaining purified oil is separated from residual solvent through distillation. Preferably, the cannabis preparation will have a Log Po/W value from five to nine.

The purified cannabis liquid is similar to honey in color and consistency. It is mixed with a carrier and any one or combination of permeation enhancer materials in selected concentrations to produce the cannabis preparation. Also, an oil may be first added to the carrier to facilitate dissolution of the cannabis components. The resultant mixture may then be optionally heated and placed within the previously described reservoir means or the mixture may be blended into a predetermined adhesive layer. Thereafter, a protective sheet may be applied and the finished assembly is packaged for storage, distribution and sale.

As noted previously, it has been found that the overall cannabis mixture is most effective with 10-90 weight percent carrier, 1-50 weight percent active cannabis, 1-90 weight percent oil and 1-15 weight percent permeation enhancer.

Examples of effective permeation enhancer materials having an HLB from 8-10 are: PEG 200 monolaurate (Mapeg 200 MO), sorbitan monolaurate (Span 20), POE myristyl ether (Lipoco-4), POE lauryl alcohol (Ethosperse LA-4), POE lauryl ether (Brij 30), POE sorbitan monooleate (Glycosperse 0-5), octyphenoxypoly (ethyleneoxy)ethanol (Igepal CA 420), linear alcohol ethoxylate (Rexonic N4), mono and diglycerides with polysorbate 80 (Tandem 8), nonyl phenol ethoxylate (Alkasurf NP-4), alkylaryl polyether ethanol (Triton X-363M), N,N-dimethyl amide (Mallcomid M 8-10).

Examples of effective permeation enhancer materials having an HLB from 11-14 are: PEG 400 monooleate (Alkamuls 400-MO), polyoxyaryl ether (Syn Fac 8210), POE oleyl alcohol (Ethosperse OA-9), PEG 600 monooleate (Alkamuls 600-MO), POE sorbitan monooleate (Atlas G8966T), PEG 400 monolaurate (Lipopeg 4-L), POG lauryl alcohol (Emthox 5967) and nonylphenoxypoly-(ethyleneoxy)ethanol (Igepal CO-720).

Examples of permeation enhancer materials having an HLB from 15-28 are: nonyl phenol ethoxylate (Alkasurf NP-15), castor oil ethoxylate (Sandoxylate C-32), ethoxylated cocomonoglyceride (Varonic LI-63), oleylalcohol condensed with ethylene oxide (Volpo-20), modified oxyethylated straight chain alcohol (Plurafac C-17), ethoxylated lanolin alcohol (Polychol 40), nonylphenyl ethoxylate (Alkasurf NP-30), polyethylene 100 stearyl ether (Brij 700), PEG 6000 monooleate (Kesso Polyethylene Glycol Esters), ethoxylated polyoxypropylene glycols (Alkatronic PGP 23-7) and ethoxylated polyoxypropylene glycols (Alkatronic PGP 23-8).

Examples of adhesive materials that may also function as a matrix to carry the active cannabis and enhancer preparations are acrylic adhesives from 3M such as 9871 Cotran neutral function pharmaceutical grade transfer adhesive, 9872 Cotran acid function pharmaceutical grade transfer adhesive and PSA 55236 acrylate copolymer. Useful acrylic adhesives from National Starch and Chemical Products are Duro-Tak 87-2516, 87-2677 and 87-2196. Other effective adhesives are polyisobutylene/light mineral oil:Oppanol B100/B10 blend 1:2 (BASF), Bio-PSA Q7-2920, 355 Medical adhesive (Dow Corning), polystryene-polybutadiene block copolymer/mineral oil:Kraton thermoplastic elastomers (Shell Corp.) and hydrogel:Methocel products (Dow Chemical).

Dosage and release information of the cannabis patches described herein are—

1) The extended period of time for cannabis administration is from 5-8 hours through 15 days.
2) The area of intact skin through which the cannabis is administered may range from about 2.54 to 7.62 square centimeters.
3) The rate at which cannabis may be administered may range from about 0.01 to 10 micrograms per square centimeter per hour.

Some examples of patch can be a cannabis patch with a rate-controlling membrane, a cannabis patch with adhesive matrix reservoir, and a cannabis patch with a foam preform as a reservoir.

Method of Use

In a further aspect of the present disclosure, it is provided a method of use, the method comprises applying to a patient in need thereof a raw cannabis composition that comprises a cannabis biomass dispersed in a transdermal carrier, and the cannabis biomass composition comprises an anesthetically effective amount of a cannabinoid acid compound and is substantially free from the cannabinoid acid compound in its decarboxylated form.

FIG. 6 shows an example of the method disclosed herein.

In some embodiments of the disclosed method, optionally in combination with any or all of the various embodiments disclosed herein, the cannabis biomass further comprises one selected from the group consisting of terpenes, flavonoids and combinations thereof.

In some embodiments of the disclosed method, optionally in combination with any or all of the various embodiments disclosed herein, the cannabinoid acid compound is tetrahydrocannabinolic acid (“THCA”) and the decarboxylated form is tetrahydrocannabinol (“THC”).

In some embodiments of the disclosed method, optionally in combination with any or all of the various embodiments disclosed herein, the transdermal carrier comprises an oil or an amount of water

In some embodiments of the disclosed method, optionally in combination with any or all of the various embodiments disclosed herein, the transdermal carrier further comprises an enhancer.

In some embodiments of the disclosed method, optionally in combination with any or all of the various embodiments disclosed herein, the raw cannabis composition is in a formulation of skin patch.

In some embodiments of the disclosed method, optionally in combination with any or all of the various embodiments disclosed herein, the biomass is in a particulate form, a pressed form, or a grinded form.

In some embodiments of the disclosed method, optionally in combination with any or all of the various embodiments disclosed herein, the patient suffers from medical condition associated with acute or chronic pain.

As used herein, the term “medical condition” refers to any medical condition that elicits a pain that can be treated or ameliorated by topical medication.

### Examples

A TheraTri composition was made according to the present disclosure and used on a subject. TheraTri is composed of a biomass having combination of non-decarboxylated, non-synthesized cannabinoids (CB), THCA (tetrahydrocannabinolic acid), CBDA (cannabidiolic acid) and CBCA (cannabichromene acid).

While the present disclosure has been described in terms of preferred embodiments, it will be appreciated by one of ordinary skill that the spirit and scope of the disclosure is not limited to those embodiments, but extend to the various modifications and equivalents as defined in the appended claims.

Further, changes may be made in the construction, operation and arrangement of the various parts, elements, steps and procedures described herein without departing from the spirit and scope of the disclosure as described in the following claims.

I claim:

1. A raw cannabis composition, comprising a cannabis biomass dispersed in a transdermal carrier, wherein the cannabis biomass composition comprises an anesthetically effective amount of a cannabinoid acid compound and is substantially free from the cannabinoid acid compound in its decarboxylated form, and wherein the cannabinoid acid compound comprises a major cannabinoid acid compound that is tetrahydrocannabinolic acid (“THCA”) and a minor cannabinoid acid compound other than THCA.

2. The raw cannabis composition according to claim 1, further comprising one selected from the group consisting of terpenes, flavonoids and combinations thereof.

3. The raw cannabis composition according to claim 1, wherein the minor cannabinoid compound is selected from the group consisting of cannabidioloic acid (“CBDA”), and cannabichromenic acid (“CBCA”), and wherein the decarboxylated form is selected from the group consisting of tetrahydrocannabinol (“THC”); cannabidiol, and cannabichromene.

4. The raw cannabis composition according to claim 1, wherein the transdermal carrier comprises an oil or an amount of water.

5. The raw cannabis composition according to claim 1, wherein the transdermal carrier further comprises an enhancer.

6. The raw cannabis composition according to claim 1, which is in a formulation of skin patch.

7. The raw cannabis composition according to claim 1, wherein the biomass is in a particulate form, a pressed form, or a grinded form.

8. A method of forming a raw cannabis composition, comprising:
providing a cannabis biomass, mixing a transdermal carrier with the cannabis biomass, and
forming a transdermal formulation of the raw cannabis composition, wherein the cannabis biomass composition comprises an anesthetically effective amount of a cannabinoid acid compound and is substantially free from the cannabinoid acid compound in its decarboxylated form,
wherein the cannabinoid acid compound comprises a major cannabinoid acid compound that is tetrahydrocannabinolic acid (“THCA”) and a minor cannabinoid acid compound other than THCA.

9. The method according to claim 8, wherein the cannabis biomass further comprises one selected from the group consisting of terpenes, flavonoids and combinations thereof.

10. The method according to claim 8, wherein the minor cannabinoid compound is selected from the group consisting of cannabidioloic acid (“CBDA”), and cannabichromenic acid (“CBCA”), and wherein the decarboxylated form is selected from the group consisting of tetrahydrocannabinol (“THC”); cannabidiol, and cannabichromene.

11. The method according to claim 8, wherein the transdermal carrier comprises an oil or an amount of water.

12. The method according to claim 8, wherein the transdermal carrier further comprises an enhancer.

13. The method according to claim 8, wherein the raw cannabis composition is in a formulation of skin patch.

14. The method according to claim 8, wherein the biomass is in a particulate form, a pressed form, or a grinded form.

15. A method, comprising applying to a patient in need thereof a raw cannabis composition that comprises a cannabis biomass dispersed in a transdermal carrier, wherein the cannabis biomass composition comprises an anesthetically effective amount of a cannabinoid acid compound and is substantially free from the cannabinoid acid compound in its decarboxylated form, and wherein the cannabinoid acid compound comprises a major cannabinoid acid compound that is tetrahydrocannabinolic acid (“THCA”) and a minor cannabinoid acid compound other than THCA.

16. The method according to claim 15, wherein the cannabis biomass further comprises one selected from the group consisting of terpenes, flavonoids and combinations thereof.

17. The method according to claim 15, wherein the minor cannabinoid compound is selected from the group consisting of cannabidioloic acid (“CBDA”), and cannabichromenic acid (“CBCA”), and wherein the decarboxylated form is selected from the group consisting of tetrahydrocannabinol (“THC”); cannabidiol, and cannabichromene.

18. The method according to claim 15, wherein the transdermal carrier comprises an oil or an amount of water.

19. The method according to claim 15, wherein the transdermal carrier further comprises an enhancer.

20. The method according to claim 15, wherein the raw cannabis composition is in a formulation of skin patch.

21. The method according to claim 15, wherein the biomass is in a particulate form, a pressed form, or a grinded form.

22. The method according to claim 15, wherein the patient suffers from medical condition associated with acute or chronic pain.
